(19) United States
(12) Patent Application Publication
(10) Pub. No.: US 2023/0218291 A1
(43) Pub. Date: Jul. 13, 2023

(54) EDGE TO EDGE REPAIR OF THE MITRAL VALVE

(71) Applicant: Ventrimend, Inc, Lake Forest, CA (US)

(72) Inventors: David Zarbatany, Lake Forest, CA (US); Ricardo Roman, Lake Forest, CA (US); Jesus Flores, Lake Forest, CA (US)

(73) Assignee: Ventrimend, Inc, Lake Forest, CA (US)

(21) Appl. No.: 18/106,460

(22) Filed: Feb. 6, 2023

Related U.S. Application Data
(63) Continuation-in-part of application No. 18/072,476, filed on Nov. 30, 2022.
Application No. 18/072,476 is a continuation-in-part of application No. 17/887,475, filed on Aug. 14, 2022.
(60) Provisional application No. 63/306,966, filed on Feb. 4, 2022.
(60) Provisional application No. 63/233,138, filed on Aug. 13, 2021.

(51) Int. Cl. A61B 17/04; A61B 17/00; A61F 2/24

(52) U.S. Cl. CPC A61B 17/0469; A61B 17/0467; A61B 17/00234; A61F 2/2412; A61B 2017/00243; A61B 2017/047

(57) ABSTRACT

The present invention describes systems and methods for treating mitral valve regurgitation. The treatment includes a systems and method of modifying the mitral valve by attaching a device to each leaflet and pulling them toward each other to stop mitral valve regurgitation.

### CROSS-REFERENCE TO RELATED APPLICATIONS

This application claims priority to U.S. Provisional Patent Application No. 63/306,966, filed Feb. 4, 2022, and is a continuation-in-part of U.S. patent application Ser. No. 18/072,476, filed Nov. 30, 2022, which is a continuation-in-part of U.S. patent application Ser. No. 17/887,475, filed Aug. 14, 2022, which claims priority to U.S. Provisional Patent Application No. 63/233,138, filed Aug. 13, 2021, the entire disclosures of which are incorporated by reference herein.

### FIELD

The present invention relates generally to the field of surgery, and more specifically, to repair of a mitral valve.

### BACKGROUND

The heart has four valves that keep blood flowing in the correct direction. The valves include the mitral valve, tricuspid valve, pulmonary valve and aortic valve. Each valve has flaps (leaflets or cusps) that open and close once during each heartbeat. Sometimes, the valves don't open or close properly, disrupting the blood flow through your heart to your body.

The mitral valve lies between the left atrium and the left ventricle. Normally, the mitral valve prevents blood flowing back into the left atrium from the left ventricle. When the mitral valve becomes leaky, it's called mitral valve regurgitation. Mitral valve regurgitation is a condition in which the mitral valve leaflets do not close tightly, allowing blood to flow backward through the mitral valve each time the left ventricle contracts. A leaking mitral valve allows blood to flow in two directions during the contraction. Some blood flows from the ventricle through the aortic valve and some blood flows back into the atrium. Leakage can increase blood volume and pressure in the left atrium. The increased pressure can increase pressure in the veins leading from the lungs to the heart (pulmonary veins). If regurgitation is severe, increased pressure may result in congestion (or fluid build-up) in the lungs. Because of this, the heart has to work harder than it should to get blood out to the body.

Accordingly, there is a need for systems and methods that provide solutions to repair mitral valve regurgitation. The present invention is directed toward systems and methods for treating these problems.

### SUMMARY

The present invention describes systems and methods for treating mitral valve regurgitation. The treatment includes a systems and method of modifying the mitral valve by attaching a device to each leaflet and pulling them toward each other to stop mitral valve regurgitation.

### BRIEF DESCRIPTION OF THE DRAWINGS

FIG. 1 is a sectional view of the heart anatomy.

FIGS. 2-11 are sectional views showing the different steps and tools to repair mitral valve leaflets.

FIG. 12 is a view of a repaired mitral valve.

FIGS. 13-16 show a perspective view and side views of an integrated therapy catheter configured to: clamp a leaflet, puncture the leaflet, and deliver one or more leaflet anchors to the leaflet.

FIG. 17 shows an anchor and tether coupled with a leaflet.

FIGS. 18-21 are views showing one embodiment of a “knot replacement” tool.

FIGS. 22-23 show components of a connect/disconnect feature of the “knot replacement” tool.

FIGS. 24-28 are views showing the cutter.

FIGS. 29-31 are views showing the “knot replacement” tool during use.

FIG. 32 shows the cutter during use.

FIG. 34 shows another embodiment of an integrated therapy catheter configured to: capture a leaflet, puncture the leaflet, and deliver one or more leaflet anchors to the leaflet.

FIGS. 35-45A, 45B show views of the operation of integrated therapy catheter of FIG. 34.

FIG. 46 shows the integrated therapy catheter advanced through a lumen in the steerable sheath exiting the distal end and grabbing the first leaflet.

FIG. 47 shows the integrated therapy catheter grabbing the second leaflet.

FIG. 48 shows the first anchor and second anchor attached to the first and second leaflets.

FIG. 49 shows the repaired mitral valve with the first and second leaflets closed with the first and second anchors.

FIG. 50 shows another embodiment of a double integrated therapy catheter configured to: capture both leaflets, puncture both leaflets, and deliver one or more leaflet anchors to both leaflets either simultaneously or sequentially.

FIG. 51 shows the integrated therapy catheter of FIG. 50 advanced through a lumen in the steerable sheath exiting the distal end and grabbing the first land second leaflets.

FIGS. 52-60 show another embodiment of the invention for treating mitral valve regurgitation.

### DETAILED DESCRIPTION

The present invention describes systems and methods for treating mitral valve regurgitation. The treatment includes a system and method of modifying the mitral valve by attaching a device to each leaflet and pulling them toward each other to stop mitral valve regurgitation.

A therapy catheter initially grasps the leaflets and a therapy catheter needle is used to puncture through the leaflets. A therapy catheter is used to deliver leaflet anchors with tethers to the leaflets. The leaflet anchors are then attached to the leaflets and pulls the leaflets toward each other. The leaflets are fixed in the desired position.

The steps include a therapy catheter directed to a first mitral valve leaflet. Through a double steerable sheath system. The “outer sheath”/transseptal sheath is placed across the intra-atrial septum from the RA to the LA. The “inner steerable”/guiding catheter is placed through the outer sheath and deflected to set a trajectory to the target location for the leaflet anchor.

The therapy catheter, internal to the inner steerable, depth is adjusted and the first leaflet is grasped.

The therapy catheter includes a cannulated needle used to puncture the first leaflet.

A first leaflet anchor and first tether are then delivered through the cannulated needle to the distal/LV side of the first leaflet. The first anchor then expands and contacts the distal/LV side of the first leaflet. The first tether is exposed as the catheter is removed.

A second leaflet anchor is delivered to the second leaflet. The second tether is exposed.

The first and second tethers are exposed through the outer steerable catheter at the groin are inserted into the “knot replacement” tool.

The knot replacement is positioned at the leaflet, the tethers are tensioned, and the knot replacement is engaged and released, leaving the tethers exposed at the groin.

The Tether Cutting Catheter follows the tethers to the knot replacement tool and are cut.

The therapy catheter initially grasps the leaflets and a therapy catheter needle is used to puncture through the leaflet. A therapy catheter is used to deliver leaflet anchors (buttons) with tethers to the leaflets. The leaflet anchors are then attached to the leaflets and pulls the leaflets toward each other, creating a coaptation area for the 2 leaflets. Once the leaflets are in the desired position. The is directed to the leaflet anchor (button) targeted location.

The Therapy Catheter depth is adjusted and the leaflet is grasped.

The Therapy Catheter needle is punctured through the leaflet to the Left Ventricle side of the leaflet.

The leaflet button is delivery and the tether is exposed as the Catheter is removed.

An opposing leaflet anchor is delivery and the tether is exposed.

The two exposed tethers exiting from the groin sheath are inserted into the “knot replacement” tool.

The knot replacement is positioned at the leaflet, the tethers are tensioned, and the knot replacement is engaged and released

The Tether Cutting Catheter follows the tethers to the knot replacement tool and are cut

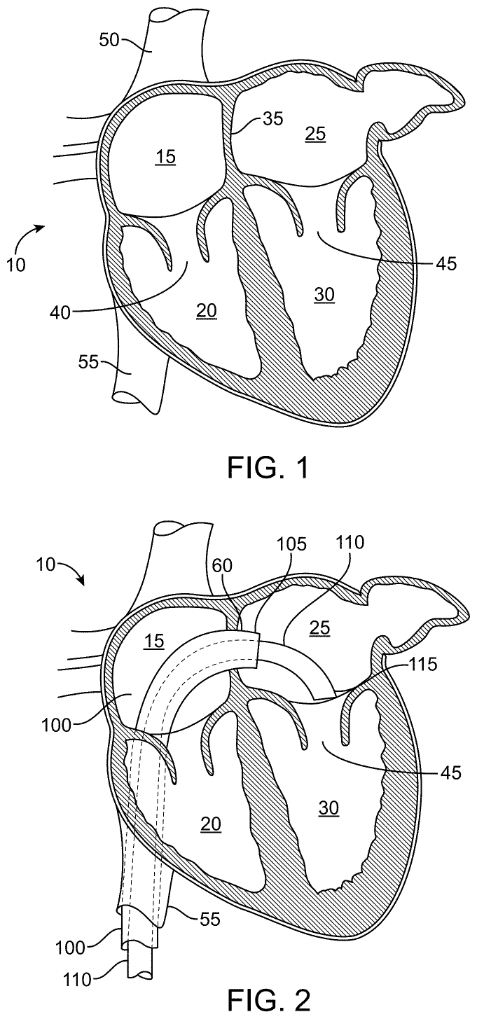

FIG. 1 is a sectional view of a normal heart anatomy 10. The heart includes four chambers, including a right atrium 15, a right ventricle 20, a left atrium 25 and a left ventricle 30. The right atrium 15 and left atrium 25 are separated by the atrial septum 35. A tricuspid valve 40 allows one way blood to flow from the right atrium 15 into the right ventricle 20. A mitral valve 45 allows one way blood to flow from the left atrium 25 into the left vertical 30.

Blood enters the right atrium 15 from the superior vena cava 50 and the inferior vena cava 55 blood vessels. The blood flows into the right atrium 15, through the tricuspid valve 40 into the right ventricle 20. Blood then flows from the right ventricle 20 into the pulmonary arteries to the lungs. Once through the lungs, the blood flows through the pulmonary veins back to the heart and into the left atrium 25. The blood from the left atrium 25 flows through the mitral valve 45 into the left ventricle 30 and out of the heart through the aortic valve to the ascending aorta.

FIG. 2 shows delivery of a delivery system 100, such as an outer steerable/transseptal sheath, that is advanced through the vascular system and into the heart 10 to the LA. The delivery system 100 enters in the femoral vein and is advanced through the vascular system to the heart and into the right atrium 15. The delivery system 100 then goes through the atrial septum 35 to left atrium 25. This can be done by puncturing a hole 60 in the wall of the atrial septum 35 between the right atrium 15 and the left atrium 25, or through a hole 60 of a patent foramen ovale or atrial septal defect, if present. A second steerable sheath 110, such as an inner steerable/guide, is inserted through the delivery system 100 and exits the distal end 105 in the LA. The second steerable sheath 110 then sets the trajectory toward the mitral valve 45. Once in the left atrium 25, the distal end 115 of the second steerable sheath 110 is advanced to the mitral valve 45.

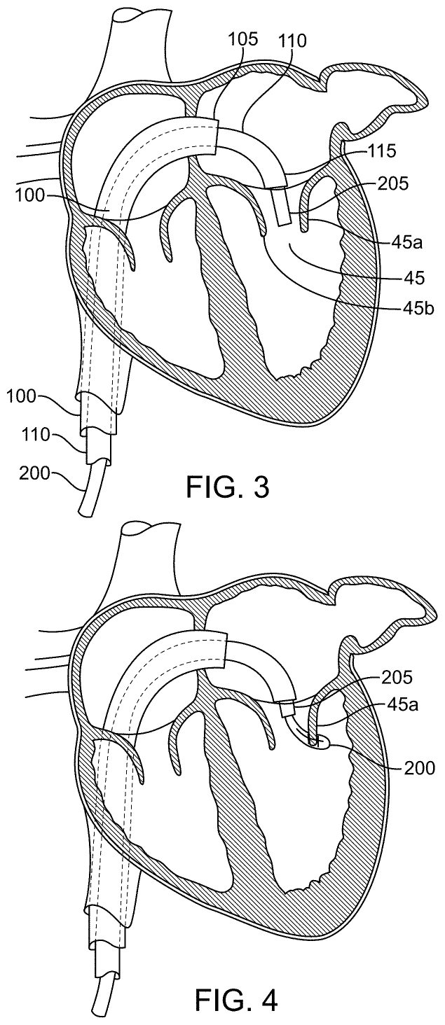

FIG. 3 shows an Inner steerable guide catheter 200 that may have an integrated therapy catheter 205 advanced through a lumen in the Guide Catheter 100. The integrated therapy catheter 205 exits the distal end 105 of the lumen.

FIG. 4 shows the integrated therapy catheter 205 advanced through the mitral valve 45 into the left ventricle 30. The integrated therapy catheter 200 depth is adjusted and a first leaflet 45a is grasped, such as the leaflet to the left ventricle (LV) side. Once grasped, a needle 220 within the integrated therapy catheter 205 advances distally and punctures the first leaflet 45a.

The needle 220 is advanced through the leaflet and the integrated therapy catheter 205 then delivers a first leaflet anchor 300 having an attached tether 305 through the puncture in the first leaflet 45a. The first leaflet anchor 300 expands or unfolds and contacts a distal side of the first leaflet 45a.

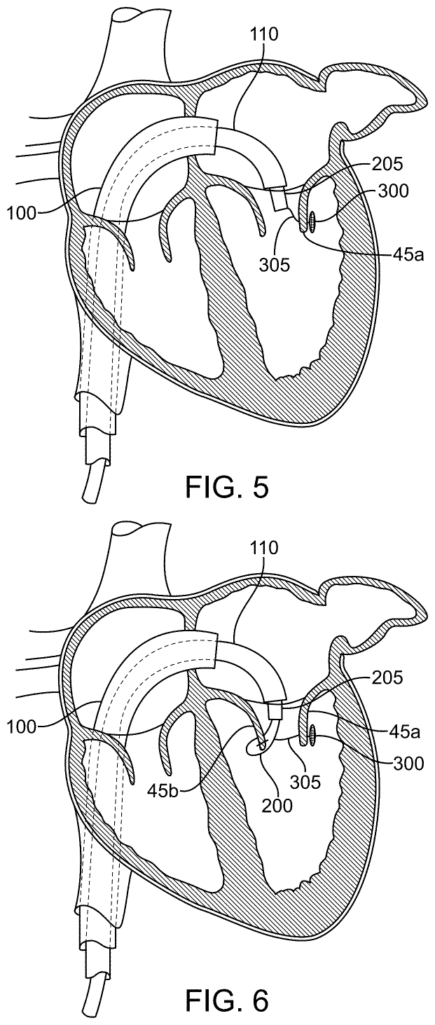

After the first leaflet anchor 300 is secured, the integrated therapy catheter 205 is withdrawn from the first leaflet 45a, shown in FIG. 5, exposing the first tether 305 attached to the first anchor 300.

FIG. 6 shows the integrated therapy catheter 205 advanced to the opposite second leaflet 45b and the depth is adjusted and the second leaflet 45b is grasped. Once grasped, the needle 220 within the integrated therapy catheter 205 advances distally and punctures the second leaflet 45b.

The integrated therapy catheter 205 then delivers a second leaflet anchor 310 having a second tether 315 through the puncture in the second leaflet 45b. The second leaflet anchor 310 is expanded or unfolds and contacts the distal side of the second leaflet 45b.

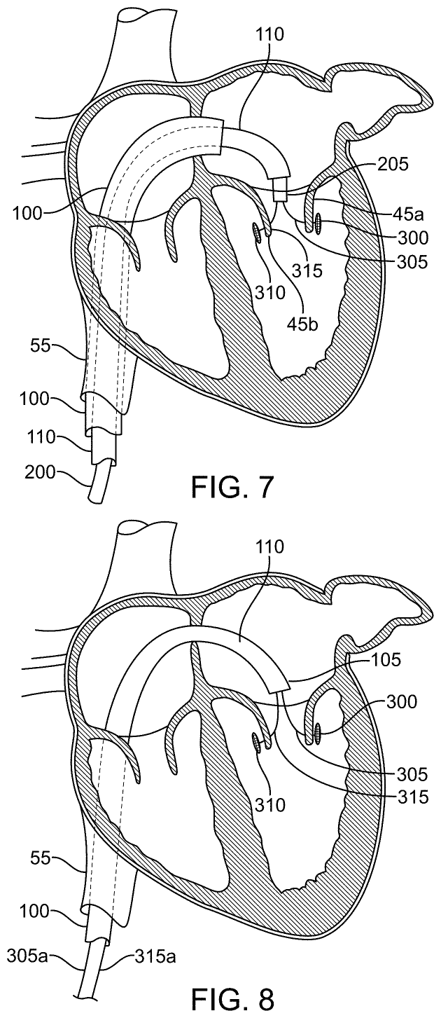

After the second anchor 310 is secured, the integrated therapy catheter 205 is withdrawn from the second leaflet 45b, shown in FIG. 7, exposing the second tether 315 attached to the second anchor 310.

The integrated therapy catheter 205 is then withdrawn from the lumen of the outer steerable catheter 100. After withdrawal, the proximal ends of first and second tethers 305a, 315a extend from the proximal end 110 of the lumen of the outer steerable catheter 100, shown in FIG. 8.

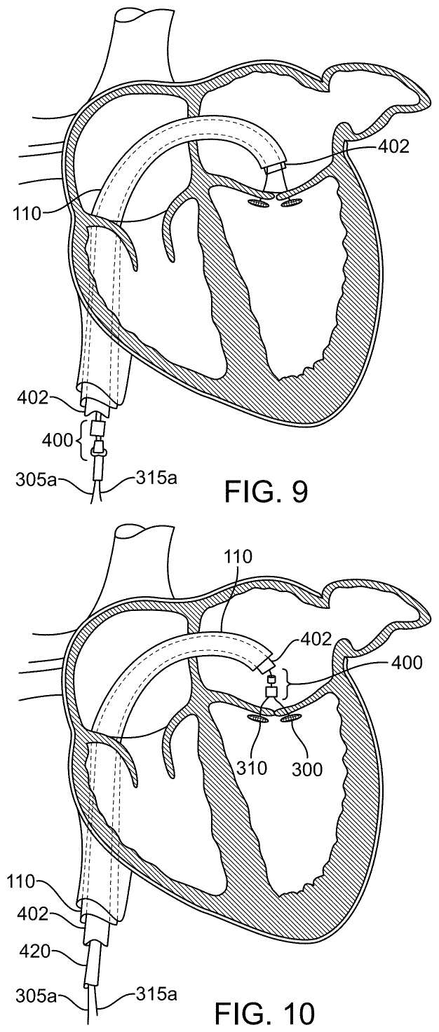

FIG. 9 shows the proximal end of the first and second tethers 305a, 315a inserted into a “knot replacement” tool 400. A knot replacement delivery catheter 402 is then inserted into the lumen of the Outer Steerable Catheter 100 and uses the first and second tethers 305, 315 as a “rail” to guide it to the mitral valve 45.

FIG. 10 shows the knot replacement tool 400 in the desired position and the tethers first and second tethers 305, 315 are tensioned so that the edges of the first and second leaflets 45a, 45b are brought together for coaptation. The knot replacement tool 400 then ties the first and second tethers 305, 315, locking them in place. The knot replacement delivery shaft 420 is detached from the knot replacement tool 400 and then withdrawn from the lumen of the outer steerable catheter 100.

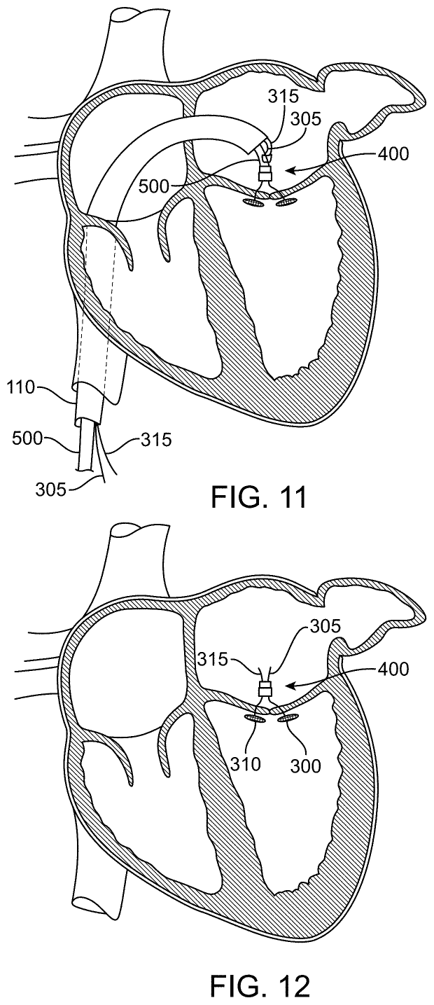

FIG. 11 the proximal end of the first and second tethers 305, 315 inserted into a tether cutter 500. A cutter delivery catheter 502 is inserted into the lumen of the outer steerable catheter 100. The cutter delivery catheter 502 and cutter 500 follow the first and second tethers 305, 315 to the locking cap 505 and locking screw 510. The cutter 500 then cuts the first and second tethers 505, 515 and the cutter delivery catheter 502 withdrawn from the guide catheter 100.

The outer steerable catheter 100 is then withdrawn from the body. If a hole in the septum needs to be repaired, an ASD closure device may be used to close the hole.

FIG. 12 shows the repaired mitral valve 45 with the first and second leaflets 45a, 45b closed with the first and second anchors.

### Integrated Therapy Catheter

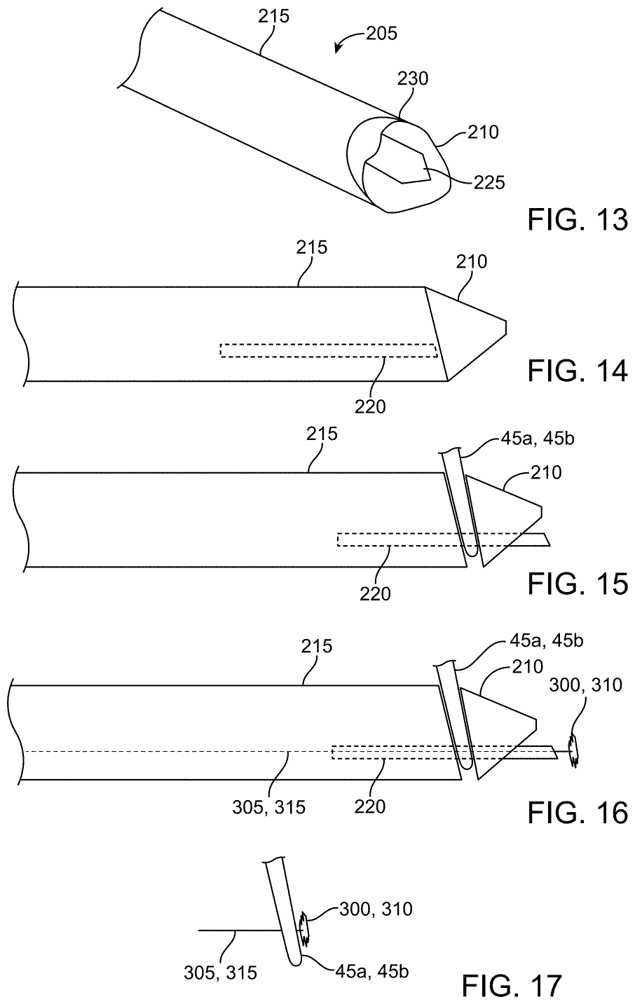

FIGS. 13-16 show a perspective view and side views of an integrated therapy catheter 205 configured to: clamp a leaflet, puncture the leaflet, and deliver one or more leaflet anchors to the leaflet through utilizing the integrated steerable sheath to actively deflect into the proper orientation and position. The integrated therapy catheter 205 includes a tissue engagement portion 210 coupled to the distal end of the body portion 215. In the embodiment shown, the tissue engagement portion 210 is a distal clamp portion 210 slidably coupled to the proximal body portion 215. The distal clamp portion 210 is configured to slide distally, and the leaflet 45a, 45b is positioned between the distal clamp portion 210 and the proximal body portion 215. The distal clamp portion 210 then moves proximally and the leaflet 45a, 45b engages distal and proximal clamp surfaces and is clamped between them.

A cannulated needle 220 is positioned within the proximal body portion 215 is configured to extend distally through a needle lumen 225 in the distal clamp portion 210.

Once the leaflet 45a, 45b is punctured, a leaflet anchor 300, 310 with a tether 305, 315 is delivered through the cannulated needle 220 to the distal side of the leaflet 45a, 45b. The cannulated needle 220 is then withdrawn and the distal clamp portion 210 extends distally to release the leaflet 45a, 45b. As then integrated therapy catheter 205 is removed in a downward direction and the tether moves up through the tether slot 230 and the leaflet anchor 300, 310 and tether 305, 315 stays attached to the leaflet 45a, 45b.

The leaflet anchor self-expands to a size larger than the puncture and engages the distal side of the leaflet. The tether extends proximally through the puncture and out of the body to be grasped during the procedure.

FIG. 17 shows the anchor 300, 310 and tether 305, 315 coupled with the leaflet 45a, 45b.

### “Knot Replacement” Tool

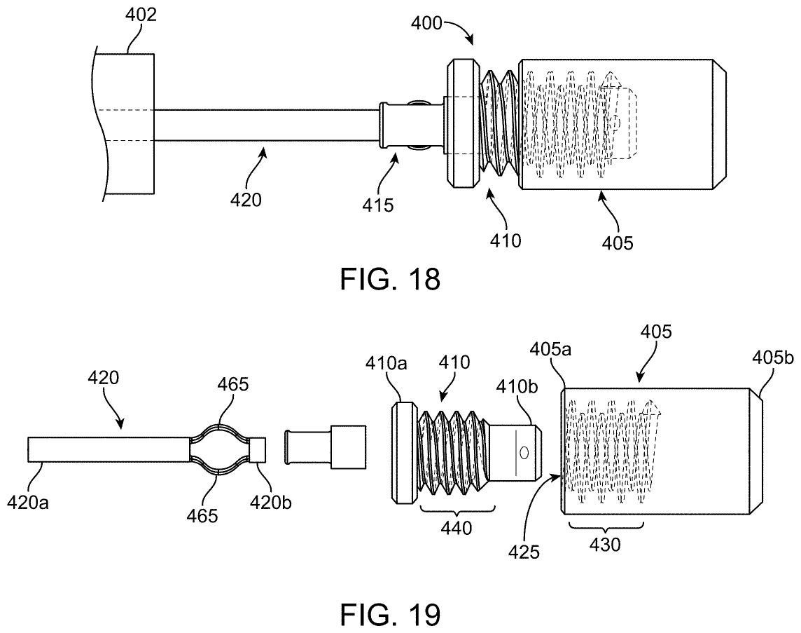

FIGS. 18-21 are views showing one embodiment of a “knot replacement” tool 400 that is configured to couple with the first and second exposed tethers 305, 315 outside the body. The first and second tethers 305, 315 are used as a “rail” to guide the “knot” replacement tool 400 to the targeted location at the first and second leaflets 45a, 45b.

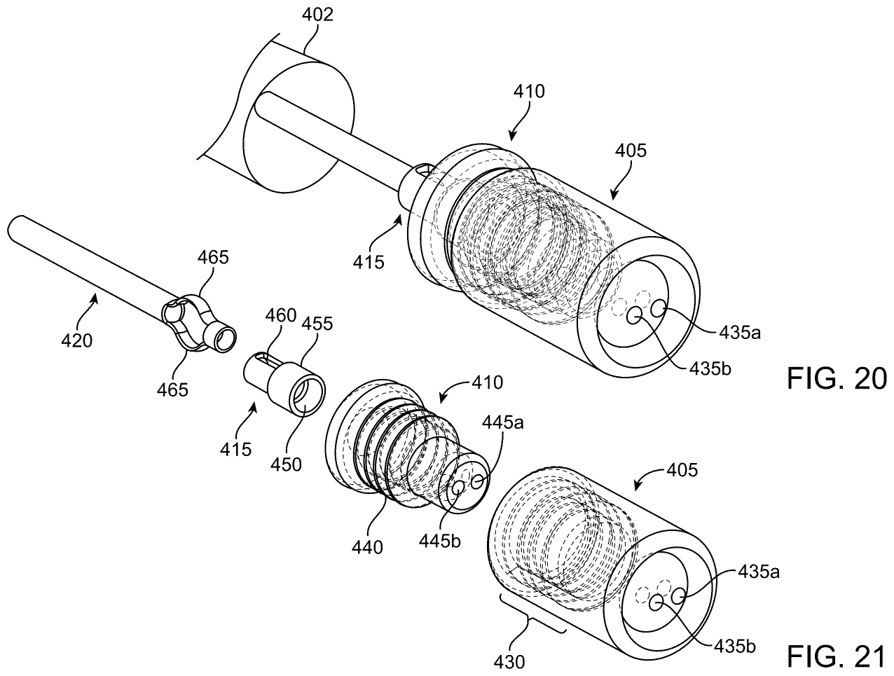

FIG. 18 is a side view of the “knot replacement” tool 400, FIG. 19 is a side exploded view of the “knot replacement” tool 400, FIG. 20 is a perspective view of the “knot replacement” tool 400, and FIG. 21 is a perspective exploded view of the “knot replacement” tool 400.

The “knot replacement” tool 400 includes a locking cap 405, a locking screw 410, a shaft coupler 415 and a delivery shaft 420.

The locking cap 405 is cylindrical in shape with a cylindrical cavity open on a proximal end 405a and closed on a distal end 405b. The cavity 425 includes an internal threaded portion 430 and the closed end includes first and second tether locking cap holes, 435a, 435b sized to receive the first and second tethers 305, 315.

The locking screw 410 includes an external threaded portion 440 and a cylindrical cavity that is open on a proximal end 410a and closed on a distal end 410b. The closed end 410b includes first and second tether locking screw holes 445a, 445b sized to receive the first and second tethers 45a, 45b. The distal end 410b is configured to be inserted into the open proximal end 405a of the locking cap 405 and the external threaded portion 440 configured to rotatingly engage with the internal threads 430 so that the locking screw 410 may be screwed into the locking cap 405.

During use, the first and second tethers 305, 315 are inserted through both the first and second tether locking cap holes 435a, 435b and the first and second tether locking screw holes 445a, 445b. As the locking screw 410 is rotated in the locking cap 405, the first and second tether locking screw holes, 445a, 445b are also rotated with respect to the includes first and second tether locking cap holes, 435a, 435b. During this rotation, the first tether 315 and second tether 315 are twisted around each other and are locked in place.

### Connect/Disconnect Feature

The shaft coupler 415 and a delivery shaft 420 are components of a connect/disconnect feature of the “knot replacement” tool 400 that are configured to couple the shaft 415 with the locking screw 410. The coupling of the shaft 420 allows torque to be applied to the locking screw 410. The design also has zero release force when the shaft 420 is uncoupled from the shaft coupler 415. This provides the ability to torque the locking screw 410 and screw the locking screw 410 into the locking cap 405. The shaft 420 can then be uncoupled without disrupting the locking screw 410 and the locking cap 405 when they are locked together.

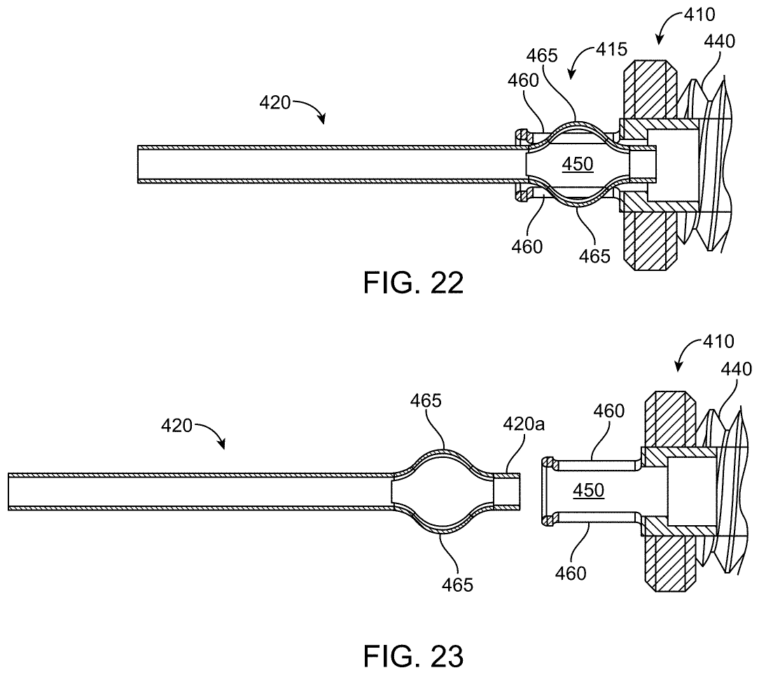

FIG. 22 is a sectional view showing the delivery shaft 420 coupled to the shaft coupler 415. The shaft coupler 415 is cylindrical in shape with a central opening 450 having a proximal portion 455 configured to be inserted into the cylindrical cavity 425 of the locking screw 410 and fixed in place. The shaft coupler 415 also includes slots 460.

The delivery shaft 420 includes a distal end 420a with engagement arms 465 having springlike properties that allow them to deflect and spring back to their original position. The distal end 420a is configured to be inserted into the into the central opening 450 of the shaft coupler 415.

The distal end of the delivery shaft 420 is sized for insertion into the central opening 450. During insertion, a curved distal portion of the engagement arms 465 contacts the shaft coupler 415 and deflects inwardly into the central opening 450 until the engagement arms 465 line up with the slots 460. Then the spring arms 465 return to their original shape and engage and lock in the slots 460. When the engagement arms 465 are coupled with the slots 460, the locking screw 410 may be rotated or torqued in the locking cap 405.

FIG. 23 is a sectional view showing the delivery shaft 420 disengaged from the shaft coupler 415. To disconnect the delivery shaft 420 from the shaft coupler 415, a removal tube is distally slid over the delivery shaft 420 until it engages the proximal end of the shaft coupler 415. Once the removal tool is engaged, the delivery shaft 420 is pulled proximally and the engagement arms 465 deflect inward, allowing removal of the delivery shaft 420 from the central opening of the shaft coupler 415.

### Cutter

FIGS. 24-28 are views showing one embodiment of a cutter 500 that is configured to couple with the first and second exposed tethers 305, 315 outside the body. The first and second tethers 305, 315 enter an opening in the distal end and exit from inner and outer side openings 520, 530. The cutter 500 then uses the first and second tethers 305, 315 as a “rail” to guide the cutter 500 to the to the targeted location proximate the “knot replacement” tool 400 at the first and second leaflets 45a, 45b.

FIG. 24 is a side view and FIG. 25 is a sectional view of a cutter 500, FIG. 26 is a distal perspective view showing more details of the cutting area of the cutter 500, FIG. 27 is a perspective view of the cutter 500, and FIG. 28 is a distal perspective view showing routing of the first and second tethers 305, 315 with the cutter 500.

The cutter 500 includes an outer cutter body 505 coupled to a braided polyimide shaft 510 and a distal stopper 515. The outer cutter body is a cylindrical tube with a side opening 520. An inner cutter body 525 is positioned within the outer cutter body 505. The inner cutter body 525 is a cylindrical tube with a side opening 530. The side openings 520 and 530 of the outer cutter body 505 and the inner cutter body 520 are positioned to provide an opening to a center lumen 555. A proximal stopper 535 which directs the tether ends through the outer and inner cutter bodies aligned side openings 520, 530. It is coupled to the distal end of the inner cutter body 525. A high torque flexible cable or torque shaft 540 is slidably positioned within the lumen of the components. The high torque cable 540 is used to rotate a thread 545 that drives the inner cutter body 525 in an axial direction 550 to scissor cut the tethers 305, 315 between the sharp edges of the inner cutter opening 530 and outer cutter opening 520. The distal end of the flexible cable 540 is positioned proximally of the side openings 520 and 530 during delivery. Once the cutter 500 is in the desired position, the tethers 305, 315 are tensioned, and the flexible cable 540 is rotated to drive the threaded cutter mechanism that moves the inner cutter 525 relative to the outer cutter 505 to cut the first and second tethers 305, 315.

### Using The “Knot Replacement” Tool

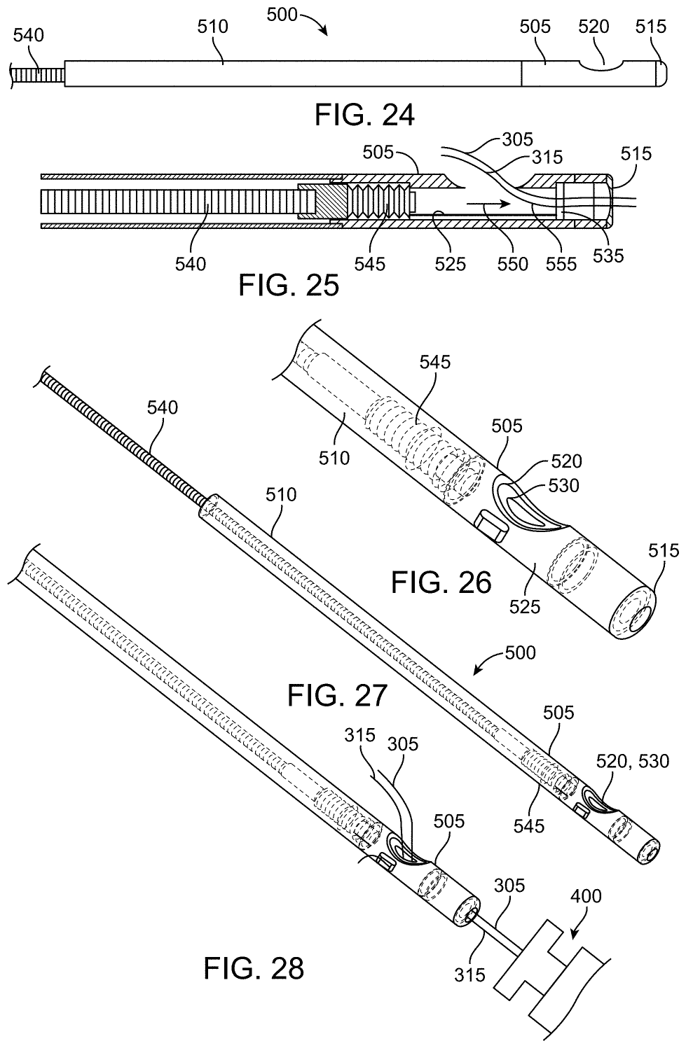

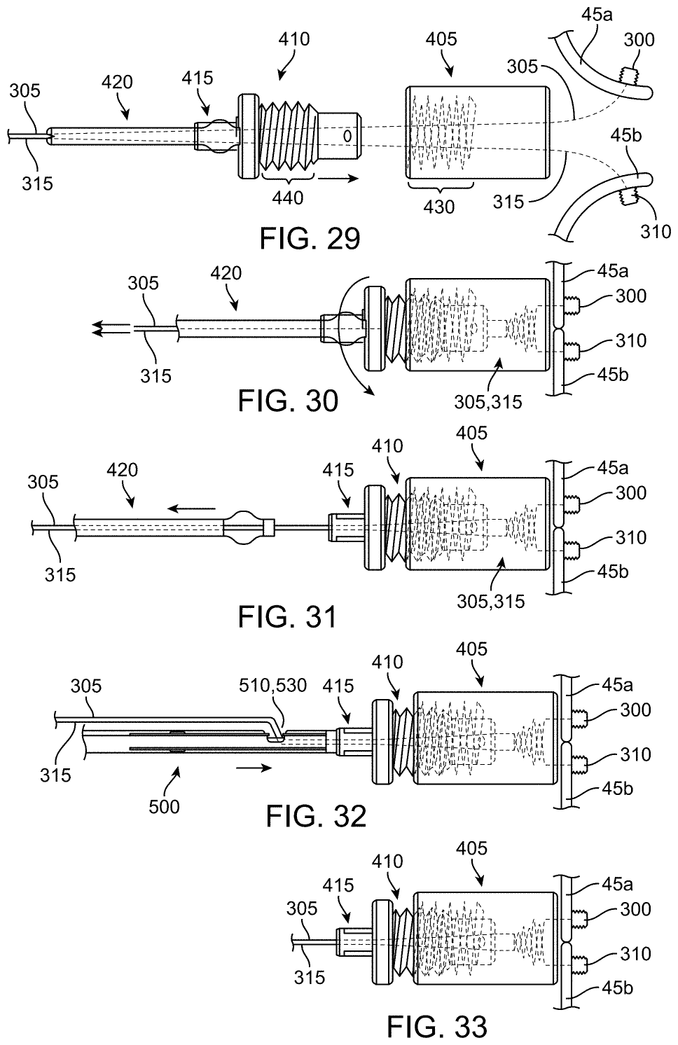

FIGS. 29-31 are views showing the “knot replacement” tool 400 engaging the first and second tethers 305, 315 attached to the first and second leaflets 45a, 45b, tensioning the first and second tethers 305, 315 to bring the first and second leaflets together 45a, 45b, and “locking” the first and second tethers 305, 315 in place.

FIG. 29 shows the first and second tethers 305, 315 inserted through the first and second tether locking cap holes, the first and second locking screw holes, through the central opening in the shaft connector and the interior lumen of the deliver shaft. The “knot replacement” tool 400 used the first and second tethers 305, 315 as a “rail” to guide the “knot” replacement tool 400 to the targeted location proximate the mitral valve 45 and first and second leaflets 45a, 45b.

FIG. 30 shows tensioning of the first and second tethers 305, 315 to pull the first and second leaflets 45a, 45b together to engage the locking cap 405. The locking screw 410 is then inserted into the locking cap and the delivery shaft 420 rotates the locking screw 410. At the same time, the first tether 305 and second tether 315 are twisted around each other and locked in place.

FIG. 31 shows the delivery shaft 420 being disconnected from the shaft coupler 415 and removed.

FIG. 32 shows a cutter 50 that also uses the first and second tethers 305, 315 as a “rail” to guide to the targeted location proximate the knot” replacement tool 400. The cutter 500 cuts the first and second tethers 305, 315 on the proximal side of the shaft coupler .415. The cutter 500 is then removed along with the cut portions of the first and second tethers 305, 315.

FIG. 33 shows the repaired first and second leaflets 45a, 45b.

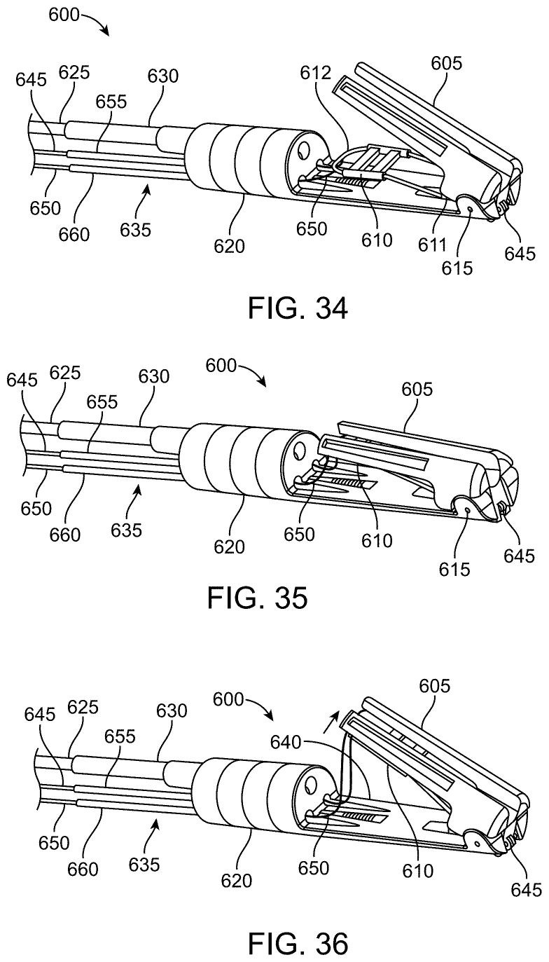

FIG. 34 shows another embodiment of an integrated therapy catheter 600 configured to: capture a leaflet, puncture the leaflet, and deliver one or more leaflet anchors to the leaflet. The integrated therapy catheter 600 includes an engagement arm 605 and a securement arm 610 rotatably coupled to a hinge 615 of a body portion 620. The engagement arm 605 and securement arm 610 are configured to rotate between an open position and a closed position. In the open position the engagement arm 605 and securement arm 610 are configured to capture the leaflet between them, and in the closed position the engagement arm 605 and securement arm 610 are configured to secure or clamp the leaflet between them.

In the embodiment shown, the securement arm 610 is made of a flexible, spring like material with a first end 611 fixedly coupled to the engagement arm 605 near the hinge 615 and a second end 612 coupled to a puller 650 that pulls and flexes the securement arm sufficiently away from the engagement arm 605 while at approximately a 45-degree angle so that the leaflet could be positioned between them. The securement arm 610 is then released and flexes back toward the engagement arm 605 so that the leaflet is clamped between them. A cannulated needle 625 is positioned within the body portion 620 and is configured to extend distally through a needle lumen 630 to puncture the leaflet.

A control mechanism 635 is coupled to the engagement arm 605 and securement arm 610 and is configured to operate or manipulate the engagement arm 605 and securement arm 610 during the leaflet capture, puncture, and release process. One end of the engagement arm 605 and securement arm 610 are joined at the hinge 615 and rotate together. The other ends are designed separate away from each other to form an opening to grasp the leaflet. This is done by flexing the securement arm 610 away from the engagement arm 605 by pulling on it with one or more pullers that are tethered to the free end. When the one or more pullers are released, the securement arm flexes back toward the engagement arm 605.

In one embodiment, the control mechanism 635 includes two mechanisms for actuation: the first mechanism is an arm actuator 645 positioned within an arm actuator lumen 655 configured to rotate the engagement arm 605 and securement arm 610 between a delivery position and a leaflet engagement position; and the second mechanism is one or more pullers 650 positioned within a puller lumen 660 configured to pull and flex the securement arm 610 away from the engagement arm 605 to capture the leaflet between them.

In some embodiments, the arm actuator 645 is a torque cable or torque shaft 645 having a first end configured to activate a cam lever in the hinge 615 to rotate the engagement arm 605 and the securement arm 610, and a second end at the handle configured to active the torque cable or torque shaft 645. In some embodiments, the arm actuator 645 is a threaded rod 645 having a first end configured to activate a cam lever in the hinge 615 to rotate the engagement arm 605 and the securement arm 610, and a second end at the handle configured to active the threaded rod 645.

In some embodiments, the one or more pullers 650 are one or more aramid fibers or Kevlar 650 having a first end tethered to the securement arm 610 and a second end at the handle to manually pull and release the securement arm 610. In some embodiments, the one or more pullers 650 are one or more sutures 650 having a first end tethered to the securement arm 610 and a second end at the handle to manually pull and release the securement arm 610.

Once the leaflet is punctured, a leaflet anchor 300 with a tether 305 is delivered through the cannulated needle 625 to the distal/under side of the leaflet. The leaflet anchor 300 is expanded. The cannulated needle 625 is then withdrawn.

The engagement arm 605 and a securement arm 610 are opened to approximately a 45-degree angle. The securement arm 610 is flexed away from the engagement arm 605 by pulling on the one or more pullers 650 to release the leaflet.

FIGS. 35-45 are views shown the operation of the integrated therapy catheter 600.

FIG. 35 shows the distal end of the integrated therapy catheter 600 in the delivery position or delivery configuration for delivery to the mitral valve through a guide catheter. In the delivery position, the engagement arm 605 and securement arm 610 are collapsed or rotated toward the body 620 and positioned within a notch 640 in the body 620.

FIG. 36 shows the engagement arm 605 and securement arm 610 in the engagement position being rotated away from the notch 640 of the body 620 by the arm actuator 645.

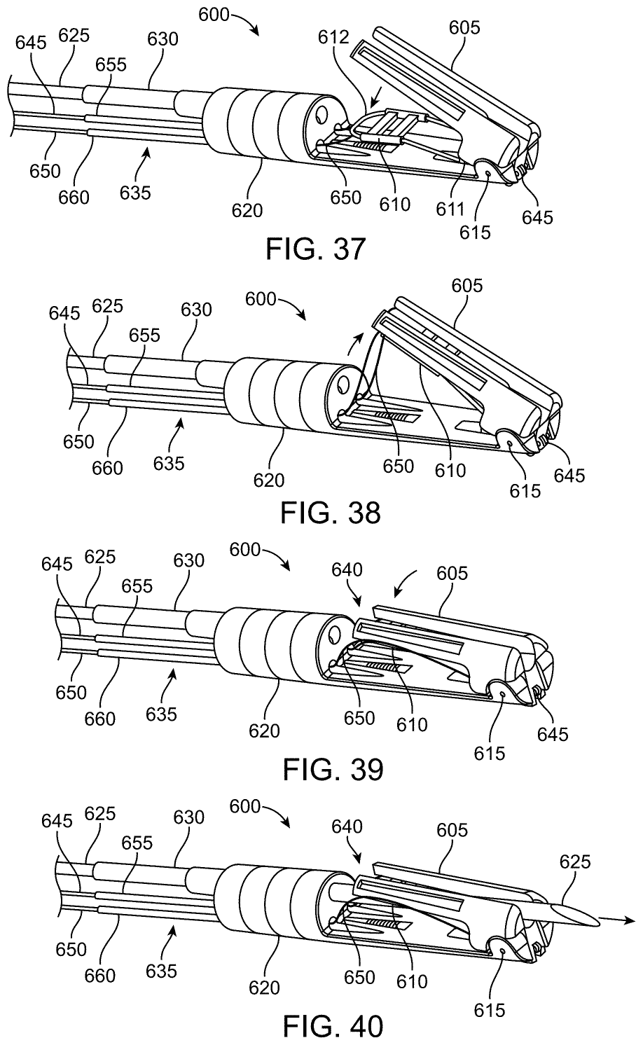

FIG. 37 shows the securement arm 610 rotated or flexed away from engagement arm 605 by the one or more pullers 650 to an open position or leaflet capture position at approximately 45 degrees.

The engagement arm 605 is then manipulated to capture the leaflet between the engagement arm 605 and securement arm 610.

FIG. 38 shows the securement arm 610 flexed upward toward the engagement arm 605 by releasing the one or more pullers 650 to a closed position or leaflet clamped position.

FIG. 39 shows the securement arm 610 and the engagement arm 605 rotated downward toward the body 620 and notch 640 with the arm actuator 645 to a leaflet puncture position.

FIG. 40 shows the cannulated needle 625 extending distally from the needle lumen 630 to puncture the leaflet.

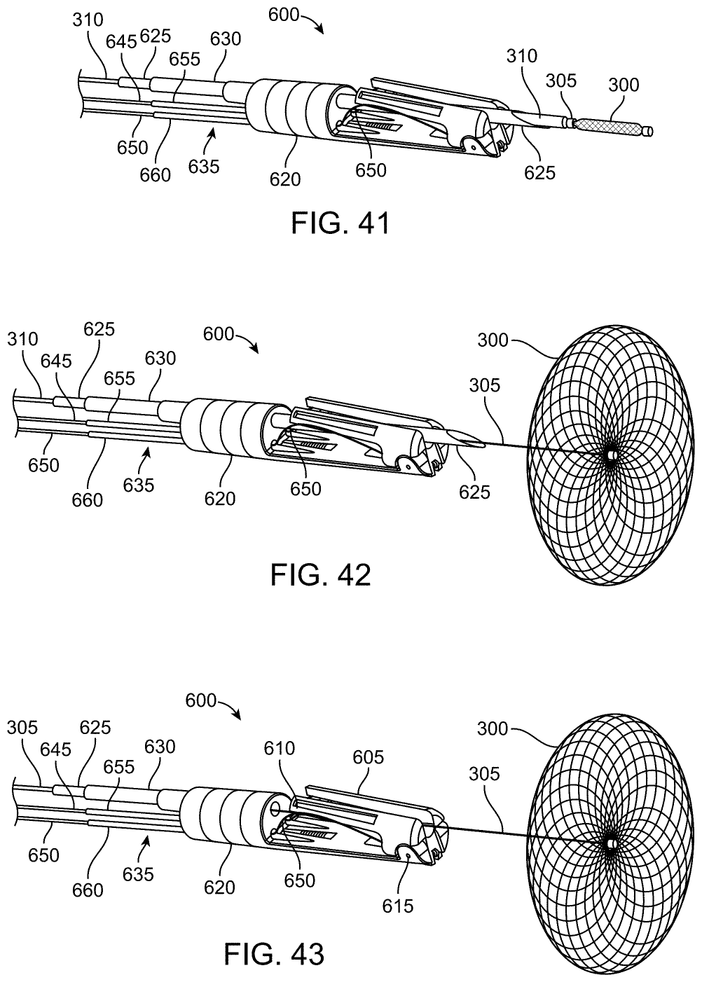

FIG. 41 shows a leaflet anchor 300 with a tether 305 within a pusher sheath 310 delivered through the cannulated needle 625 to the distal/under side of the leaflet.

FIG. 42 shows the leaflet anchor 300 expanded.

FIG. 43 shows the cannulated needle 625 withdrawn proximally into the needle lumen 630 and the pusher sheath 310 is also withdrawn.

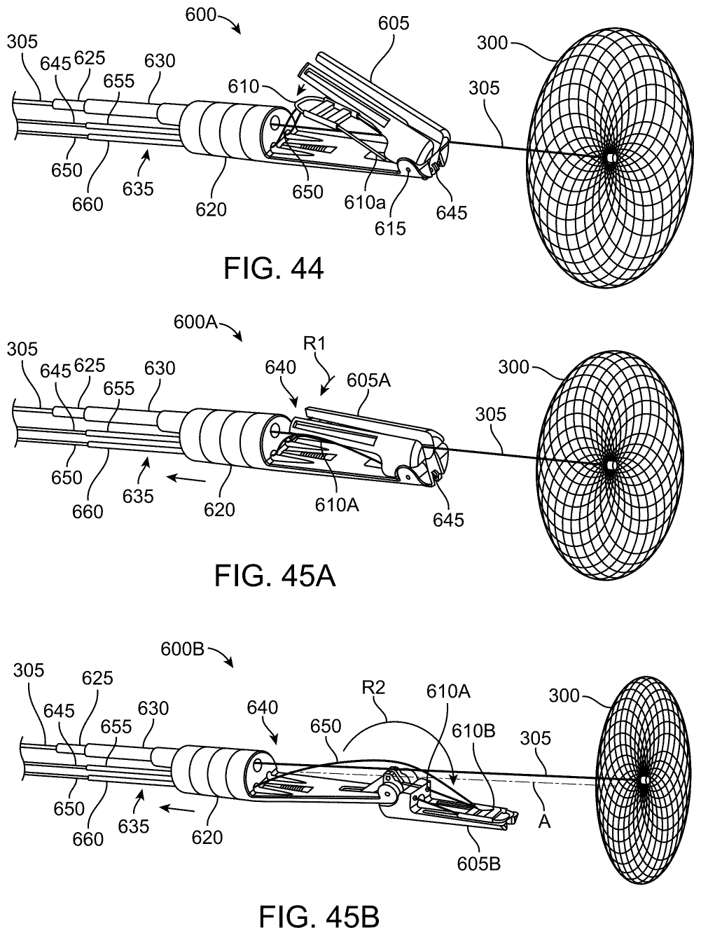

FIG. 44 shows the securement arm 610 flexed away from the engagement arm 605 with the one or more pullers 650 to the open position to release the leaflet.

FIGS. 45A and 45B show two embodiments for rotation of the engagement arm 605 and securement arm 610 for withdrawal of the integrated therapy catheter 600.

FIG. 45A shows one embodiment for the withdrawal configuration of integrated therapy catheter 600A with the engagement arm 605A and securement arm 610A rotated proximally R1 into the notch 640 for storage during withdrawn of the integrated therapy catheter 600A proximally from the leaflet anchor 300.

FIG. 45B shows another embodiment for the withdrawal configuration of integrated therapy catheter 600B with the engagement arm 605B and securement arm 610B rotated distally R2 to approximately 180 degrees or parallel to the catheter axis A during withdrawal of the integrated therapy catheter 600B proximally from the leaflet anchor 300.

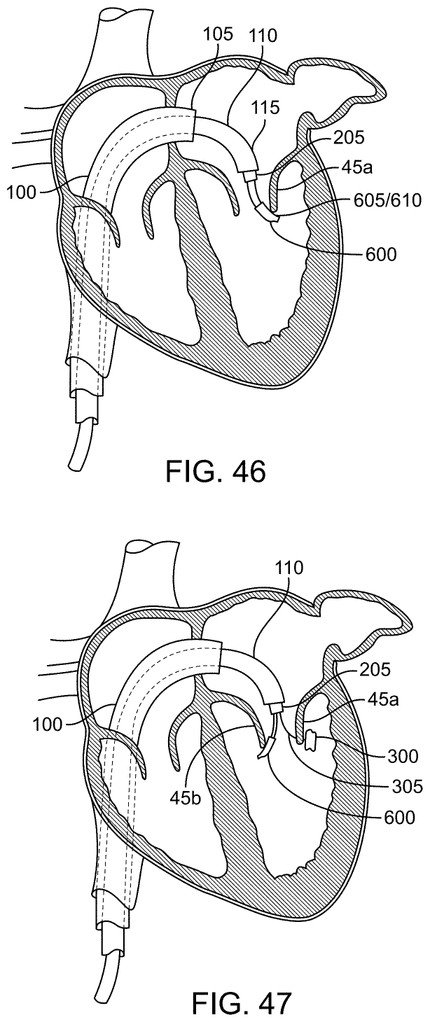

FIG. 46 shows the delivery system 100 that has been advanced through the vascular system and into the heart 10 to the LA and the steerable sheath 110 has been inserted through the delivery system 100, exiting the distal end 105 and advanced to the mitral valve 45.

The integrated therapy catheter 600 is advanced through a lumen in the steerable sheath 110 exiting the distal end 115 and advanced through the mitral valve 45 into the left ventricle 30. The securement arm 610 and the engagement arm 605 are rotate to the open position, the securement arm 610 is pulled away from the engagement arm 605, and the first leaflet 45a is grasped with the engagement arm underneath the leaflet and from the left ventricle (LV) side. Once grasped, the securement arm 610 is released to close on the engagement arm 605. Then both the securement arm 610 and the engagement arm 605 are rotated together to the closed or clamped position and the cannulated needle 625 advances distally and punctures the first leaflet 45a.

The integrated therapy catheter 600 then delivers a first leaflet anchor 300 having an attached tether 305 through the puncture in the first leaflet 45a.

FIG. 47 shows the first leaflet anchor 300 expands or unfolds and contacts a distal side of the first leaflet 45a.

After the first leaflet anchor 300 is secured, the integrated therapy catheter 600 is withdrawn from the first leaflet 45a, exposing the first tether 305 attached to the first anchor 300.

The process is then repeated for opposite second leaflet 45b. the integrated therapy catheter 600 advanced to the opposite second leaflet 45b and the second leaflet 45b is grasped securement arm 610 and the engagement arm 605. Once grasped, the needle 625 advances distally and punctures the second leaflet 45b.

The integrated therapy catheter 600 then delivers a second leaflet anchor 310 having a second tether 315 through the puncture in the second leaflet 45b. The second leaflet anchor 310 is expanded or unfolds and contacts the distal side of the second leaflet 45b.

After the second anchor 310 is secured, the integrated therapy catheter 600 is withdrawn from the second leaflet 45b, exposing the second tether 315 attached to the second anchor 310.

The integrated therapy catheter 600 is then withdrawn from the lumen of the outer steerable catheter 100. After withdrawal, the proximal ends of first and second tethers 305a, 315a extend from the proximal end 110 of the lumen of the outer steerable catheter 100.

The “knot replacement” tool 400, mounted on the distal end of the knot replacement tool delivery catheter 402, uses the first and second tethers 305, 315 as a “rail” to guide it to the mitral valve 45. The first and second tethers 305, 315 are then tensioned so that the edges of the first and second leaflets 45a, 45b are brought together forcing coaptation. The knot replacement tool 400 then ties the first and second tethers 305, 315, with the locking cap 405 and locking screw 410, locking them in place. See FIG. 10. The knot replacement delivery catheter is then removed leaving the proximal tethers intact and extending from the proximal end 110 of the lumen of the outer steerable catheter 100.

The proximal end of the first and second tethers 305, 315 inserted into the tether cutter 500. The cutter delivery catheter 502 and cutter 500 follow the first and second tethers 305, 315 to the locking cap 405 and locking screw 410. The cutter 500 then cuts the first and second tethers 505, 515 and the cutter delivery catheter 502 withdrawn from the guide catheter 100. See FIG. 11

FIG. 49 shows the repaired mitral valve 45 with the first and second leaflets 45a, 45b closed with the first and second anchors.

### Double Integrated Therapy Catheter

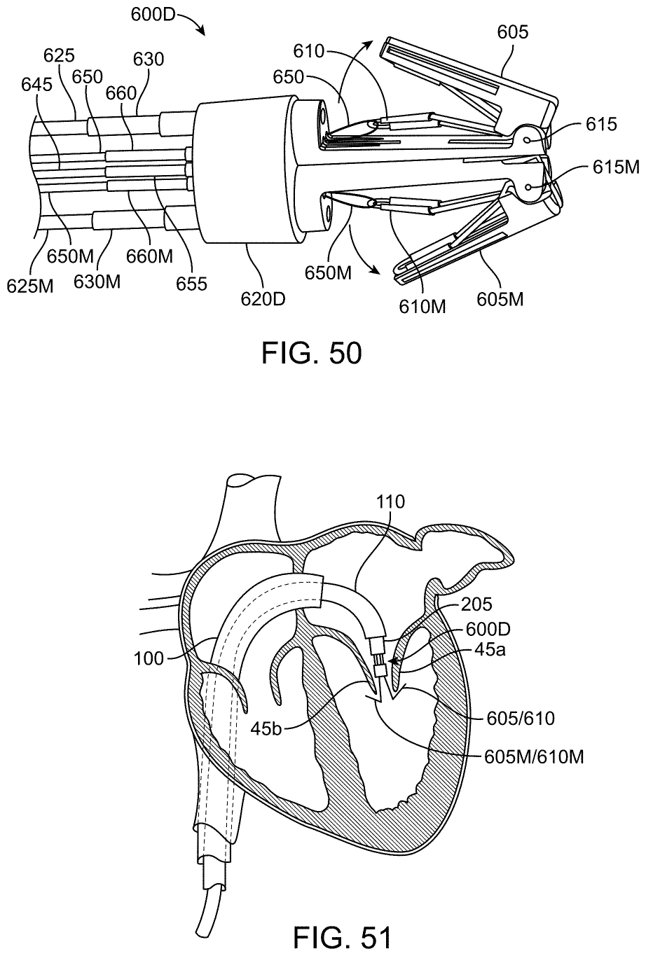

FIG. 50 shows another embodiment of an integrated therapy catheter 600D (600 Double) that includes a double-sided grasper that can grasp two leaflets either at the same time or in series and deliver two anchors and tethers (one to each leaflet). The double integrated therapy catheter 600D is similar to the integrated therapy catheter 600 but also includes a second mirror image integrated therapy catheter 600M (600 Mirror) that are coupled together at a double body portion 620D

The integrated therapy catheter 600D includes two engagement arms 605, 605M and two securement arms 610, 610M rotatably coupled 615, 615M to a double body portion 620D. The arm actuator 645 is configured to rotate the engagement arms 605, 605M and securement arms 610, 610M between a closed position to an open position. The pullers 650, 650M are configured to flex the securement arms 610, 610M away from the engagement arms 605, 605M to capture both leaflets 45a, 45b and secure or clamp the leaflets 45a, 45b between them, either one at a time or simultaneously. In the embodiment shown, the engagement arms 605, 605M are opened to approximately a 45-degree angle and the securement arms 610, 610M are flexed by the pullers 650, 650M to so that the leaflets 45a, 45b are positioned between the engagement arms 605, 605M and securement arms 610, 610M. The pullers 650, 650M are then released and the securement arms 610, 610M flex back toward the engagement arms 605, 605M so that the leaflets 45a, 45b are clamped between them. Two cannulated needles 625, 625M are positioned within the double body portion 620D and are configured to extend distally through needle lumens 630, 630M to puncture the 45a, 45b leaflets. The arm actuator 645 and pullers 650, 650M are coupled to the engagement arms 605, 605M and securement arms 610, 610M and are configured to operate or manipulate the engagement arms 605, 605M and securement arms 610, 610M together and/or separately during the leaflet 45a, 45b capture, puncture, and release process. In the embodiment shown, one arm actuator 645 is used to control both sets engagement arms 605, 605M and securement arms 610, 610M so that they open together. Other embodiments may include two arm actuators to separately control the engagement arm 605 and securement arm 610 and mirror engagement arm 605M and securement arm 610M. A first arm actuator 645 is used to actuate engagement arm 605 and securement arm 610, and a second mirror arm actuator 645M is used to actuate mirror engagement arm 605M and securement arm 610M. The leaflets 45a, 45b may both be grasped simultaneously or sequentially. Once both leaflets are grasped, the puncture and anchor delivery could be done simultaneous or sequential. The leaflets 45a, 45b are the released and the delivery system 600D retracted leaving two tethers 305 outside of the femoral vein to complete the procedure described above.

FIG. 51 shows the delivery system 100 that has been advanced through the vascular system and into the heart 10 to the LA and the steerable sheath 110 has been inserted through the delivery system 100, exiting the distal end 105 and advanced to the mitral valve 45.

The double integrated therapy catheter 600D is advanced through a lumen in the steerable sheath 110 exiting the distal end 115 and advanced through the mitral valve 45 into the left ventricle 30. The securement arm 610 and the engagement arm 605 are rotate to the open position and the first leaflet 45a is grasped. The mirror securement arm 610M and the engagement arm 605M are rotate to the open position and the second leaflet 45B is grasped. Once grasped, the securement arms 610, 610M and the engagement arms 605, 605M rotate to the closed or clamped position and the cannulated needle 625 advances distally and punctures the first leaflet 45a and the cannulated needle 625M advances distally and punctures the second leaflet 45b either simultaneously or sequentially.

The integrated therapy catheter 600M then delivers a first leaflet anchor 300 having an attached tether 305 through the puncture in the first leaflet 45a and a second leaflet anchor 310 with attached tether 315 through the puncture in the second leaflet 45b. The first leaflet anchor 300 expands or unfolds and contacts a distal side of the first leaflet 45a and the second leaflet anchor 310 expands or unfolds and contacts a distal side of the second leaflet 45b.

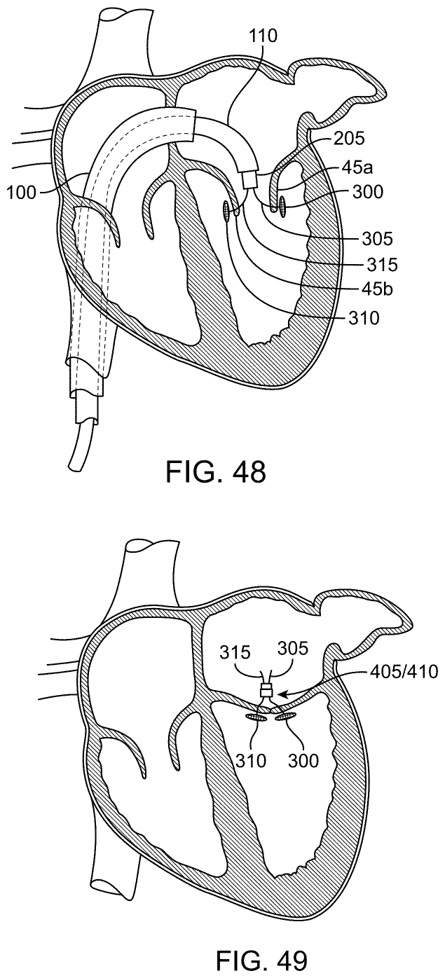

After the first and second leaflet anchors 300, 310 are secured, the integrated therapy catheter 600M is withdrawn from the first and second leaflets 45a, 45b, exposing the first and second tethers 305, 315 attached to the first and second anchors 300, 310 (see FIG. 48).

The integrated therapy catheter 600D is withdrawn from the lumen of the outer steerable catheter 100. After withdrawal, the proximal ends of first and second tethers 305a, 315a extend from the proximal end 110 of the lumen of the outer steerable catheter 100.

The “knot replacement” tool 400, mounted on the distal end of the knot replacement tool delivery catheter 402, uses the first and second tethers 305, 315 as a “rail” to guide it to the mitral valve 45. The first and second tethers 305, 315 are then tensioned so that the edges of the first and second leaflets 45a, 45b are brought together for coaptation. The knot replacement tool 400 then ties the first and second tethers 305, 315, with the locking cap 405 and locking screw 410, locking them in place. See FIG. 10. The knot replacement delivery catheter is then removed leaving the proximal tethers intact and extending from the proximal end 110 of the lumen of the outer steerable catheter 100.

The proximal end of the first and second tethers 305, 315 inserted into the tether cutter 500. The cutter delivery catheter 502 and cutter 500 follow the first and second tethers 305, 315 to the locking cap 405 and locking screw 410. The cutter 500 then cuts the first and second tethers 505, 515 and the cutter delivery catheter 502 withdrawn from the guide catheter 100. See FIG. 49 showing the repaired mitral valve 45 with the first and second leaflets 45a, 45b closed with the first and second anchors.

### Mitral Valve Repair With Pledgets

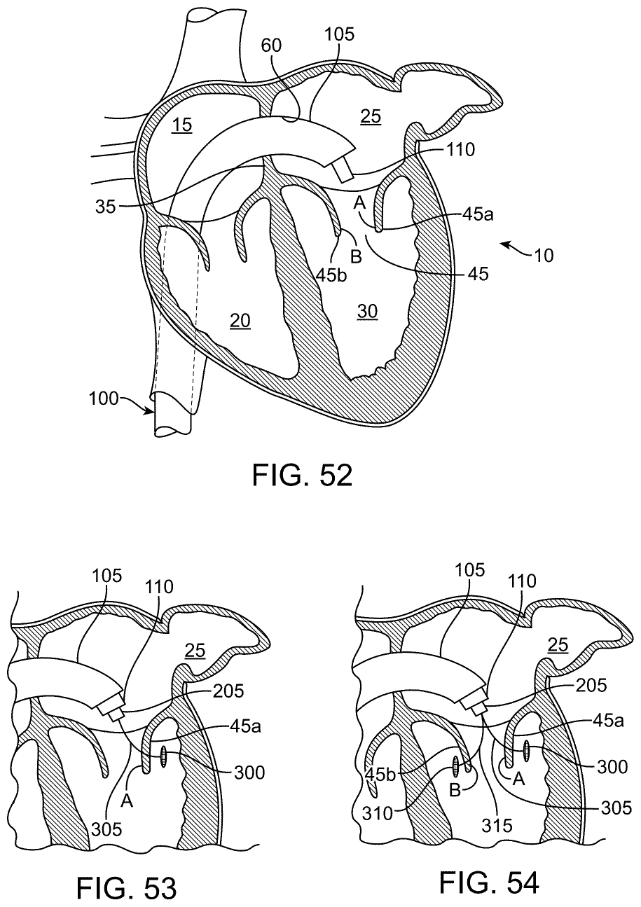

FIGS. 52-60 show another embodiment of the invention for treating mitral valve regurgitation. The treatment includes systems and methods of modifying the mitral valve by attaching a device to each leaflet and pulling them toward each other to stop mitral valve regurgitation.

FIG. 52 shows a delivery system 100 having a guide catheter 105 that enters in the femoral vein and is advanced through the vascular system to the heart 10 and into the right atrium. The guide catheter 105 then goes through the atrial septum 35 to left atrium 25. This can be done by puncturing a hole 60 in the wall of the atrial septum 35 between the right atrium 15 and the left atrium 25, or through a hole 60 of a patent foramen ovale or atrial septal defect, if present. A steerable sheath 110, such as an inner steerable/guide, is inserted through the guide catheter 105 and exits the distal end in the left atrium 25 and sets the trajectory toward the mitral valve 45.

FIG. 53 shows an integrated therapy catheter 205 advanced through a lumen in the guide catheter 105 or the steerable sheath 110. The integrated therapy catheter 205 is advanced to the mitral valve 45 and a valve engagement structure 210 extends from the distal end.

The valve engagement structure 210 engages a first leaflet 45a at location A to hold the integrated therapy catheter 205 in place while a cannulated needle 220 punctures the first leaflet 45a (see FIG. 4). The integrated therapy catheter 205 then delivers a first anchor 300 having an attached tether 305 through location A. The first anchor 300 expands or unfolds and engages the left ventricle 30 side of the first leaflet 45a. The integrated therapy catheter 205 is then disengaged from the first leaflet 45a.

FIG. 54 shows the integrated therapy catheter 205 advanced to a second leaflet 45b of the mitral valve 45, the valve engagement structure 210 engages the second leaflet at location B to hold the integrated therapy catheter 205 in place while a cannulated needle 220 punctures the second leaflet 45b of the mitral valve 45 at location B (see FIG. 6). The integrated therapy catheter 205 then delivers a second anchor 310 having a second tether 315 through location B in the mitral valve 45. The second anchor 310 is expanded or unfolds and engages the left ventricle 30 side of the second leaflet 45b.

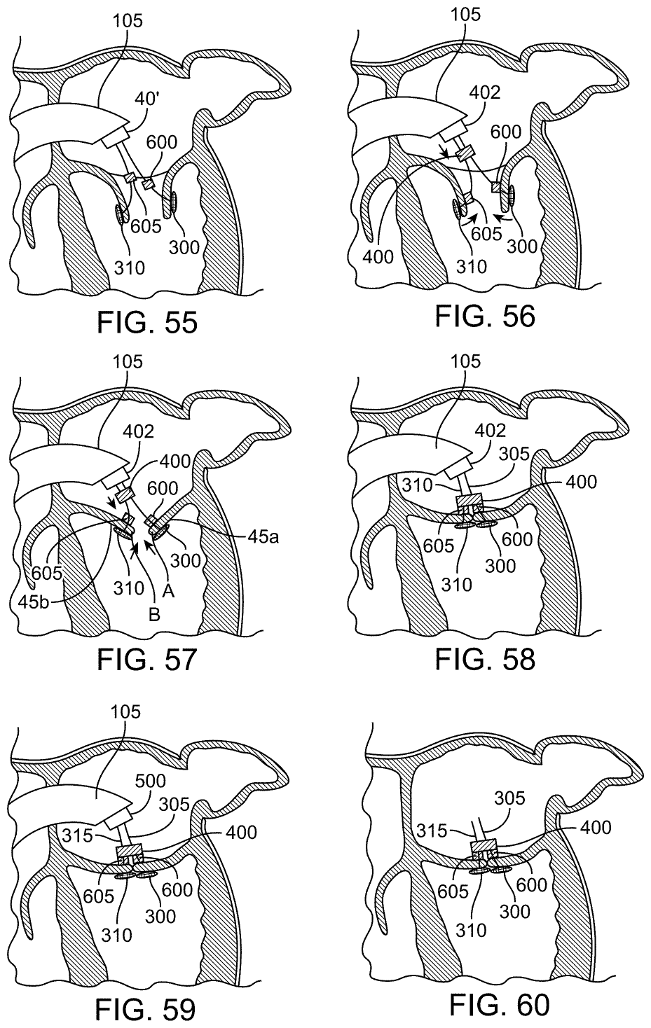

FIG. 55 shows the first anchor 300 with the first tether 305 attached to the first leaflet 45a at location A, and the second anchor 310 with second tether 315 attached to the second leaflet 45b of the mitral valve 45 at location B. A first pledget 600 is slid over the proximal end of the first tether 305 and a second pledget 605 is slid over the proximal end of the second tether 315. The first and second pledgets 600, 605 are inserted through the lumen of the guide catheter 105 and advanced toward locations A and B.

FIG. 56 shows the first pledget 600 positioned on the first leaflet 45a at location A, opposite the first anchor 300, and the second pledget 605 positioned on the second leaflet 45b of the mitral valve 45 at location B, opposite the second anchor 310. A knot replacement delivery catheter 402 is inserted into the lumen of the guide catheter 105. The proximal end of the first and second tethers 305, 315 are inserted into a knot replacement tool 400. The knot replacement tool 400 uses the first and second tethers 305, 315 as a rail to guide it towards locations A and B of the mitral valve 45.

FIG. 57 shows the knot replacement tool 400 advancing over the first and second tethers 305, 315 toward locations A and B of the mitral valve 45. The advancing movement of the knot replacement tool 400 tensions the first and second tethers 305, 315 and pulls the first and second anchors 300, 310 toward each other, reducing the distance between location A and location B. The first and second pledgets 600, 605 are used to provide support on the leaflets 45a, 45b to prevent the first and second anchors 300, 310 from tearing through the leaflet tissue.

FIG. 58 shows the final position of the first and second anchors 300, 310 and the distance between location A and location B. In this position, the first leaflet 45a is next to the second leaflet 45b. The knot replacement tool 400 is rotated to tie the first and second tethers 305, 315 together, locking them in place. The knot replacement delivery shaft 402 is then detached from the knot replacement tool 400 and withdrawn.

FIG. 59 shows a tether cutter 500 sliding along the first and second tethers 305, 315 through the lumen of the guide catheter 105 and following the first and second tethers 305, 315 to the knot replacement tool 400. The tether cutter 500 then cuts the first and second tethers 305, 315 near the knot replacement tool 400. The tether cutter 500 is then withdrawn and finally the catheter 105 is withdrawn from the body.

FIG. 60 shows the repaired mitral valve 45 with the knot replacement tool 400 in the locked position with the first and second pledgets 600, 605 positioned against the leaflets tissue on the first and second tethers 305, 315, locking the first and second leaflets 45a, 45b in a closed position.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A system for edge-to-edge repair of a mitral valve comprising:
an integrated therapy catheter configured to engage a first leaflet and a second leaflet of a mitral valve having:
a puncture device configured to:
puncture a first hole in the first leaflet; and
puncture a second hole in the second leaflet;
a delivery device configured to:
deliver a first leaflet anchor through the first hole, the first leaflet anchor having a first tether with distal end configured to engage the distal side of the first leaflet;
deliver a first pledget coupled to the first tether to the first hole;
deliver a second leaflet anchor through the second hole, the second leaflet anchor having a second tether with distal end configured to engage the distal side of the second leaflet.
deliver a second pledget coupled to the second tether to the second hole; and
a “knot replacement” tool configured to tension the first and second tethers to bring the first and the second leaflet edges together and lock the first and second tethers together.

2. The system of claim 1, wherein the “knot replacement” tool includes:
a locking cap having first and second tether locking cap holes, the first and second tethers being inserted through the first and second locking cap holes; and
a locking screw having first and second tether locking screw holes, the first and second tethers being inserted through the first and second locking screw holes;
wherein the locking screw is configured to be screwed into the locking cap, with the first and second tether locking screw holes and the first and second tether locking cap holes rotated in opposite directions so that the first tether and second tether are twisted around each other and are locked in place.

3. The system of claim 2, wherein “knot replacement” tool further includes a detachable delivery shaft coupled with the locking screw.

4. The system of claim 1, further comprising a cutter device configured to cut the first and second tethers.

5. The system of claim 4, wherein cutter device includes:
an outer body with an outer body side opening; and
an inner body with an inner body side opening slidably positioned within the outer body, the inner body side opening and the outer body side opening are positioned to provide an opening to a center lumen,
wherein the first and second tethers enter a distal end of the center lumen and exit out through the inner body side opening and outer body side opening, and
axial movement of the inner body in relation to the outer body side opening closes the opening to the center lumen and scissor cuts the first and second tethers.

6. The system of claim 1, wherein the integrated therapy catheter further includes a clamping device configured to clamp a leaflet, the clamping device having a distal clamp portion slidably coupled to a proximal body portion, wherein the distal clamp portion is configured to slide distally to position the leaflet between the distal clamp portion and the proximal body portion and then the distal clamp portion is configured to slide proximally and the leaflet is clamped between the distal clamp portion and the proximal body portion.

7. The system of claim 6, wherein puncture device includes a cannulated needle positioned within the proximal body configured to extend distally to puncture a hole in the leaflet.

8. The system of claim 7, wherein delivery device is configured to deliver a leaflet anchor through the cannulated needle.

9. The system of claim 1, wherein distal ends of the first and second tethers are self-expanding and configured to expand larger than the first and second holes.

10. The system of claim 1, wherein the puncture device configured to puncture the first hole proximate a first leaflet edge and puncture a second hole proximate a second leaflet edge.

11. A system for edge-to-edge repair of a mitral valve comprising:
an integrated therapy catheter configured to engage a first leaflet and a second leaflet of a mitral valve having:
a puncture device configured to puncture a first hole in the first leaflet proximate a first leaflet edge and puncture a second hole in the second leaflet proximate a second leaflet edge;
a delivery device configured to:
deliver a first leaflet anchor through the first hole to a distal side of the first leaflet, the first leaflet anchor having a first tether with a self-expanding distal end configured to expand larger than the first hole and engage the distal side of the first leaflet;
deliver a first pledget coupled to the first tether to the proximal side of the first hole;
deliver a second leaflet anchor through the second hole to a distal side of the second leaflet, the second leaflet anchor having a second tether with a self-expanding distal end configured to expand larger than the first hole and engage the distal side of the second leaflet;
deliver a second pledget coupled to the second tether to the second hole; and
a “knot replacement” tool configured to couple with the first tether and the second tether and tension the first tether and the second tether to bring first leaflet edge and the second leaflet edge together, and lock the first tether and tether second tether together.

12. The system of claim 11, wherein the integrated therapy catheter further includes a clamping device configured to clamp a leaflet, the clamping device having a distal clamp portion slidably coupled to a proximal body portion, wherein the distal clamp portion is configured to slide distally to position the leaflet between the distal clamp portion and the proximal body portion and then the distal clamp portion is configured to slide proximally and the leaflet is clamped between the distal clamp portion and the proximal body portion.

13. The system of claim 12, wherein puncture device includes a cannulated needle positioned within the proximal body configured to extend distally to puncture a hole in the leaflet.

14. The system of claim 13, wherein delivery device is configured to deliver a leaflet anchor through the cannulated needle.

15. The system of claim 11, wherein the “knot replacement” tool includes:
a locking cap having a cylindrical body with an internal threaded portion and a closed distal end having a first tether locking cap hole and a second tether locking cap hole; and
a locking screw having a cylindrical body with an external threaded portion and a closed distal end having first and second tether locking screw holes;
wherein the locking screw is configured to be screwed into the locking cap, with the first and second tether locking screw holes and the first and second tether locking cap holes rotated in opposite directions so that the first tether and second tether are twisted around each other and are locked in place.

16. The system of claim 15, wherein “knot replacement” tool further includes a detachable delivery shaft coupled with the locking screw.

17. The system of claim 11, further comprising a cutter device configured to cut the first and second tethers, the cutter device includes:
an outer body that is a cylindrical tube with outer body side opening; and
an inner body slidably positioned within the outer body, the inner body is a cylindrical tube with an inner body side opening, the inner body side opening and the outer body side opening are positioned to provide an opening to a center lumen,
wherein the first and second tethers enter the distal end of the center lumen and exit out of the center lumen through the inner body side opening and outer body side opening, and
axial movement of the inner body scissor cuts the first and second tethers between the edges of the inner body side opening and outer body side opening.

18. A method for edge-to-edge repair of a mitral valve comprising:
puncturing a first hole in a first leaflet proximate a first leaflet edge and puncturing a second hole in a second leaflet proximate a second leaflet edge;
delivering a first leaflet anchor through the first hole to a distal side of the first leaflet, the first leaflet anchor having a first tether with a self-expanding distal end configured to expand larger than the first hole and engage the distal side of the first leaflet;
delivering a first pledget coupled to the first tether to the first hole;
delivering a second leaflet anchor through the second hole to a distal side of the second leaflet, the second leaflet anchor having a second tether with a self-expanding distal end configured to expand larger than the first hole and engage the distal side of the second leaflet; and
delivering a second pledget coupled to the second tether to the second hole; and
coupling the first tether and the second tether with a knot replacement tool;
tensioning the first tether and the second tether with the knot replacement tool to bring first leaflet edge and the second leaflet edge together; and
locking the first tether and tether second tether.

19. The method of claim 18, further comprising cutting the first and second tethers

20. The method of claim 18, wherein the “knot replacement” tool includes:
a locking cap having a cylindrical body with an internal threaded portion and a closed distal end having a first tether locking cap hole and a second tether locking cap hole; and
a locking screw having a cylindrical body with an external threaded portion and a closed distal end having first and second tether locking screw holes;
wherein the locking screw is configured to be screwed into the locking cap, with the first and second tether locking screw holes and the first and second tether locking cap holes rotated in opposite directions so that the first tether and second tether are twisted around each other and are locked in place.
